(19) United States
(12) Patent Application Publication
(10) Pub. No.: US 2023/0256129 A1
(43) Pub. Date: Aug. 17, 2023

(54) WEARABLE PERSONAL PROTECTION DEVICE

(71) Applicant: AVID BUSINESS PTY LTD (ACN: 083025003) AS TRUSTEE FOR CEDAR CREEK TRUST, South Pumpenbil, NSW (AU)

(72) Inventors: Timothy BOLAND, South Pumpenbil, NSW (AU); Thomas BOLAND, South Pumpenbil, NSW (AU)

(21) Appl. No.: 18/014,245

(22) PCT Filed: Jul. 1, 2021
(86) PCT No.: PCT/AU2021/000043

§ 371 Date: Jan. 3, 2023

(30) Foreign Application Priority Data
Jul. 1, 2020 (AU) 2020902231

(51) Int. Cl. A61L 9/14; A61M 11/00; A61M 35/00

(52) U.S. Cl. CPC A61L 9/14; A61M 11/005; A61M 35/10; A61L 2209/132; A61L 2209/111; A61M 2209/088; A61M 2205/8206; A61M 2205/0294

(57) ABSTRACT

A wearable personal protection device for protecting a wearer against airborne pathogens comprises a battery-powered electronic circuit and components for controllable operation of at least one piezoelectric vibrating mesh transducer which is configured to intermittently simultaneously generate and shoot droplets of a biocide fluid into the wearer's proximal airspace to contact and inactivate at least some of the pathogens which may be present; a reservoir for containing the biocide fluid therein; and a fluid delivery apparatus for conveying said biocide fluid to an uptake location of the piezoelectric vibrating mesh transducer.

### TECHNICAL FIELD

The present invention relates to personal protective equipment, particularly for prophylaxis against airborne pathogens, irritants, allergens and pollutants.

### Definition of Terms

The terms “shoot” and “shot” are used in this specification to mean “droplet projectiles targeted at an object and or an area”.

The term “biocide fluid” is used in this specification to mean “a pure biocide, a fluid or a liquid containing a biocide”.

The term “intercept” is used in this specification to mean “to approach and strike a target object and or area”.

The term “droplet” in this specification means “a cohesive mass of fluid of diameter <1000 μm”.

The term “aerosol” in this specification means “at least one cohesive mass of matter of diameter or length 10 μm or less.

The term “respiratory droplet” is used in this specification to mean “a small mass of any diameter which includes at least one of a) water, b) respiratory mucus; generated within and exhaled from the respiratory system of a person, c) saliva”.

The term “wearer's airspace” in this specification means “the wearer's proximal breathing airspace: a volume of air having a rear boundary centred between the nose and mouth and which rear boundary includes the surface of the face; generally, the volume from which a human draws minute volume breath and exhales into during a normal moderately active day, which volume in this specification≈12 litres”. See also FIG. 4A.

The term “biocide” is used in this specification to mean “a substance which inactivates or destroys pathogens, irritants, and allergens on contact”.

The term “pathogen” in this specification means “any infectious microorganism, and any irritant or any allergen”.

The term “electrical circuit” in this specification means “electrically conductive tracks together with electrical components required to provide a said electrically activated functionality”.

The term “wicking element” is used in this specification to mean “a material which conveys a fluid by capillary action”.

The term “app” in this specification means “a computer application software which executes on mobile devices: smartphones, smartwatches, computer tablets and the like”.

The term “artificial intelligence technology (AI)” in this specification means “computer software which has predefined goals and which processes data to make data-based decisions, learn and take actions available to it to most effectively realize its goals”.

The statement “at least one of” in this specification means “at least one of, some of, all of, or any combination of listed items a), b), c), d), e), f), etc.”.

The phrase “and or” is used in this specification to mean “at least one of”.

The term “mucosa” in this specification means “epithelial and other cells which airborne pathogens may infect to gain entry to the body, including the mucosa of the of the respiratory system, eyes, Lacrimal punctum, Lacrimal canals, nose, and mouth”.

Throughout this specification, unless the context requires otherwise, the word “comprise” or variations such as “comprises” or “comprising”, will be understood to imply the inclusion of a stated integer or group of integers but not the exclusion of any other integer or group of integers.

### BACKGROUND

The Present Invention pertains to Personal Protective Equipment (PPE) suitable for use by all people, for personal protection against disease-causing airborne viruses, bacteria, and fungi. Said pathogens include but are not limited to SARS-CoV-2, Influenza A, Mycobacterium tuberculosis, Aspergillus spp, and Streptococcus pneumoniae which cause serious diseases of high morbidity rates; further including pathogens which cause disease of generally lower and low morbidity, but which cause much suffering and can develop into serious disease and cause death: Rhinoviruses, Bordetella pertussis, Varicella-zoster virus, Measles morbillivirus, Respiratory Syncytial Virus. The invention also protects against airborne fungal spores and pollen grains. Respectively, the diseases caused are: newly emerged lethal disease COVID-19, Flu, Tuberculosis, Aspergilloma, Bacterial Pneumonia, common cold, Whooping cough, Chickenpox, Measles, Bronchiolitis and Viral Pneumonia, Toxic pneumonitis, hypersensitivity pneumonitis, Allergic rhinitis (Hay fever).

Most people suffer a respiratory infection several times per year. Consequently, each year tens of billions of respiratory infections occur. The majority of cases are sub-clinical (asymptomatic), but hundreds of millions of cases are more serious and manifest clinical symptoms which progress to serious, debilitating, life-threatening disease. Every year about 5 million people suffer and die of respiratory disease.

Given the high incidence of respiratory infection, and that all infected humans release a cloud of <5 μm mucus droplets, when coughing, sneezing, speaking and simply breathing, it is to be expected that air in environments inhabited by humans will often contain infectious airborne pathogens. Indeed, several studies have proved this so.

Much research literature evidence that specific pathogens are more prevalent and more infectious in certain environments. For example, air samples taken from medical doctor's waiting rooms and passenger aircraft cabins have been found to contain very high levels of infectious pathogens, most notably coronaviruses.

A 2014 study conducted by the Department of Virology, Moscow State University collected air samples from a healthcare-centre, a day-care centre, and aeroplanes, and found:
“Concentrations of airborne influenza viruses (A/PR/8/34 (H1N1) and A/swine/Minnesota/1145/2007 (H3N2)) were found to be quite high. Fifty percent of samples contained viruses in concentrations ranging from 5.8×10³ to 3.7×10⁴ genome copies per m³. The average concentration of the virus was 1.6±0.9×10⁴ genome copies per m³.” (Nikitin et al., 2014)

Similarly, a USA study by Prussin et al., (2015) reported on virus and bacteria levels in the air in the USA. Air samples were taken in nine locations “a classroom, a daycare center, a dining facility, a health center, three houses, an office, and outdoors” were found to contain astonishingly high levels of ^{˜}10⁵ particles m⁻³ [100,000 per m3] in each environment. They estimated the total number of virus-like particles and bacteria particles inhaled daily to be approximately 6×10⁶ each; the equivalent of 12,000,000 pathogens being inhaled each day. In addition to virus and bacteria, referencing the work of Reponen et al., (1994, Prussin et al., (2015) agree “humans [also] inhale between 60 and 60,000 fungal spores daily.”

Notably, Prussin et al., (2015) expressed surprise to discover as many bacteria as viruses were suspended in the air; expecting that bacteria, some 50-70 times larger than viruses, would be far less numerous. Evidently misled by settling times in still air, the researchers expected bacteria would not remain suspended in air for more than a few minutes. However, given that even large rod-shaped Mycobacterium tuberculosis bacteria rarely exceed 4 μm in length and 0.5 μm in diameter, and given that aerosol droplets of up to 5 μm diameter are known to remain suspended in mildly turbulent air indefinitely, it is to be expected that bacteria too will remain suspended for hours, perhaps days.

Furthermore, various air temperatures, humidity levels and barometric pressures are known to increase the prevalence, viability and infectiousness of various pathogens. Coronaviruses for example are more prevalent, more infectious and survive longer in air temperature ranges of 0-5° C. and low (30%-40%) or high (70%) relative humidity.

It is also known that various barometric pressures, relative humidity and temperatures coincide with the release of various forms of pollen and fungal spores which become airborne and are known to cause allergic rhinitis (hay fever), asthmatic symptoms and other respiratory infections in susceptible individuals. And, a wide variety of airborne fungi, most of which are saprobe fungi, are known to cause Otomycosis or fungal otitis which also infects the outer ear canal.

COVID-19 has brought to the forefront the need to find new and more effective infectious respiratory disease transmission control measures. Declared a pandemic 11 Mar. 2020 by the World Health Organization, to the end of June 2021, the WHO reported 164 million cases and 3.4 million deaths. The medical research literature evidence that the highly infectious SARS-CoV-2 virus is airborne.

It is known that all airborne pathogens require mucosa epithelial cells as the entry point to the body. Vulnerable areas include the mucosa of the eyes, mouth, throat, and the vast surface area of the upper and lower airways. The most serious symptoms and diseases result from an infection of the alveoli, deep within the lungs.

### Sources of Airborne Pathogens

The majority of infectious respiratory diseases are caused by viruses and bacteria. Fungal infections may result from fungi spores released into the air from both indoor and outdoor fungi. However, in the case of airborne viral and bacterial infection, the source is generally humans. Not only is the respiratory system infected by airborne pathogens, but the infected respiratory system is the major source of airborne pathogens which infect the respiratory system of other humans who inhale airborne pathogens exhaled by infected persons.

All humans generate respiratory droplets. Expiratory events including breathing, speaking, laughing, sneezing, coughing are known to generate and release respiratory droplets into the air. Droplets range from 0.6 μm to 1000 μm in diameter. Numbers released vary much from person to person, in the range of 1-50 respiratory droplets emitted per second; as high as 200 per second in individuals known as super-emitters. Given that average tidal breathing is 15 breaths per minute, each exhalation the average person emits into the air 4-200 droplets. A super-emitter may release up to 800 respiratory droplets each exhalation.

When released, large respiratory droplets (10 μm to 1000 μm) generally settle to the floor in seconds and minutes due to gravity acting on their greater mass and inertia. If inhaled before they fall to the floor, they generally leave airstream and deposit in the upper areas of the respiratory tract; become trapped in mucous and are removed from the body via excretions and by the Mucociliary Escalator. Infections that do result are generally upper respiratory tract infections and are generally less severe than lower respiratory tract infections.

Small <10 μm respiratory droplets may remain suspended in normally turbulent room air indefinitely and may be inhaled for up to many hours, perhaps days after their release into the air. Further, this small size range is known to comprise <99% of all respiratory droplets released. Further, having little mass and therefore negligible inertia, if inhaled they may remain in airstream much longer than large droplets. Many travel deep into the lungs and deposit in the lower respiratory tract. Smaller droplets of <5 μm diameter may travel to the alveoli and deposit on highly sensitive alveolus walls. Lower respiratory tract infections produce more severe symptoms. Alveoli infections are known to result from lower pathogen loads, produce the most severe symptoms, and may develop into life-threatening respiratory disease, and cause death.

Medical research literature increasingly evidences that pathogen carrying <10 μm droplets and particles are likely responsible for the majority of transmissions of all airborne respiratory diseases.

Furthermore, studies have found that high numbers of infective pathogens and <10 μm respiratory droplets are continually present, suspended in the air of the home, workplace, public, and natural environments. People are at risk of respiratory tract infection in all environments.

It is to be noted that the higher the density of people in a specific space, the greater the number of mucus droplets are released into the air and the greater the probability of the presence of high pathogen loads in that airspace.

All the above factors come together such that airborne pathogens cause hundreds of millions of cases of clinical infection each year. Each year tens of millions of people young and old suffer distressing, debilitating disease. Each year some 5 million people needlessly suffer and die of respiratory diseases transmitted by airborne pathogens.

Whatever the pathogen, its naked presence in the air or presence in airborne mucus droplets presents a grave danger to all humans breathing that air.

### Deficiencies of Existing Wearable PPE Devices

The following discussion of the background art is intended to facilitate an understanding of the present invention only. The discussion is not an acknowledgement or admission that any of the material referred to is or was part of the common general knowledge as at the priority date of the application.

A number of forms of wearable personal protective equipment (PPE) devices designed to protect wearers from inhaling airborne pathogens are known and many are commercially available. Existing forms employ at least one of two base concepts: i) filtering the wearer's air, ii) treating air before moving it on by fans or other conveyance means to deliver air to the wearer's nose and mouth region. Both concepts have inherent disadvantages.

Filtering forms include single-use respirators (simple filtration masks), reusable respirators, positive pressure respirators, and electronic forms.

Single-use Masks

The most common forms of PPE devices are simple masks of filter media adapted to fit over the wearer's mouth and nose to prevent particles in the airstream from entering the respiratory system. The filter media is retained in position by thin cords which tie at the back of the head, by elasticised bands that fit around the back of the head, or by elastic loops which fit around the ears. Kronzer et al., U.S. Pat. No. 5,307,796A (1994) teaches this form.

The filter media is most commonly a multi-layer nonwoven laminate of spun or melt blown fine plastic fibres. Generally, at least one laminate is electrostatically charged to increase filtering efficiency. Kubik & Davis U.S. Pat. No. 4,215,682A (1980) teaches a means of creating the filter media.

The general public, particularly in high population density Asian cities, commonly wear this form for protection against pollution particles and Flu viruses. Medical workers wear a variant form to protect themselves and their patients from airborne particles and droplets which may cause infection. Yavitzet, U.S. Pat. No. 5,803,075A (1998) teaches this variant.

Various occupational health authorities rate the effectiveness of these forms according to the percentage of particulates they entrap. Relatively high 95% entrapment designations include N95 (USA), FFP2 (Eu), KN95 (China), P2 (Australia/NZ), 1^{st} Class (South Korea), DS (Japan).

Although the filter material is effective at trapping particles, these forms have significant deficiencies. Firstly, the filter media traps and concentrates pathogens on its outer surface and throughout its thickness and may come to contain a high pathogen load. Breath intake thereafter may draw pathogens and pathogen containing particles from the filtering mesh into the wearer's respiratory system

Secondly, the mask itself becomes a fomite. Indeed, many occupational health authorities make it law that employers instruct employees in careful procedures for safe removal of these masks from the face; to limit the transfer of pathogens from mask to hands. Some forms add a biocide to the fibres or to an area of the mask. However, over time pathogens may build in the fibres, the biocidal effect decreases and infective pathogens may be released into the wearer's breathed air.

Thirdly, filter restriction of exhaled airflow, in masks not fitted with an exhaust valve, result in a portion of exhaled CO2 being rebreathed. Some users are known to experience mild hypoxia-induced faintness and cloudy thinking as a result.

Further, lacking an effective seal, filtering efficiency is much reduced if the mask is not well fitted around the face, particularly the difficult nose to face junction area. And if the wearer has facial hair a seal cannot be established.

Furthermore, the constant force of the elasticised straps is known to cause pressure urticaria and general inflammation at the mask to skin contact areas.

In hospitals, these masks are routinely fitted to patients suffering a respiratory illness to protect medical staff from pathogens emitted in the patient's exhalations. This retards recovery of the patient. Not having an exhaust valve, so pathogens are not vented, the patient's breathing is restricted both on exhalation and inhalation. Furthermore, because the mask is not vented, the patient rebreathes a portion of the pathogens removed by exhalation, back into their lungs.

If an infected person is fitted with a mask having an exhaust valve, infective pathogens are vented into the air, to be rebreathed by themselves, by others present, and by others who may enter that environment and breathe that air hours and days later.

Additionally, this form does not provide protection from infection via the mucus membranes and Lacrimal punctum of the eyes. It is to be noted that the Lacrimal punctum opens to Lacrimal canals which convey fluid from the eye into the nasal cavity. Infection may occur in the mucus membrane of the eyes, in the Lacrimal canals and the nasal cavity.

Furthermore, in the case of medical industry workers, medical practice often requires that staff dispose of a mask after each patient contact, or every hour or so. Medical workers may use and dispose of as many as 12 or more masks per working day and each removal carries a risk of infection.

A great many of these masks are used, disposed of and constantly resupplied. The number disposed of yearly would likely be >1.5 billion. Considering only Asia, assuming 1% of the adult public (estimated at 23.2 million) use one mask and replace it weekly, each year >1.2 billion masks would be discarded. Further assuming 1% of the world's medical workers (1% of some 59 million) use a single-use mask 3 times per day for 48 weeks, and assuming 1 million patients are fitted with one mask per day for a 3-day hospital stay, medical use would consume some 428 million masks p.a. Total masks consumed would be about 1.63 billion. This equates to about 7,365 metric tonnes of plastic manufactured and discarded. As the filter media is generally made from hydrocarbon derived polypropylene, each year about 7,365 tonnes or more of crude oil is used to make disposable masks.

Furthermore, increased public usage of disposable masks during the COVID-19 pandemic has resulted in international news reports of thousands of discarded masks littering waterways and public places. This constitutes a new pollution problem and given that many discarded masks may be contaminated with pathogens, may further spread disease.

Reusable Masks

Reusable masks are available in full and half mask forms. Full mask forms include a transparent shield to protect the wearer from infection via the mucus membranes and Lacrimal canals of the eyes. Reischel et al., U.S. Pat. No. 5,924,420 (1999) teaches this form. Reusable half-masks do not include said transparent shield. Matheson & Lowry, U.S Pat. No. 4,414,973A (1983) teaches this form.

This form generally provides a mask cup having a soft, resilient plastic edge that conforms to the contours of the wearer's face to provide a better seal. Thicker, replaceable filter media which may include an activated carbon element to trap vapours, is also provided. Although offering improved sealing, and particle filtering approaching 100%, these forms have significant deficiencies.

Firstly, the thicker filter material causes greater breathing resistance. Although generally fitted with an exhausted valve to reduce exhalation resistance, breath intake requires greater effort than disposable masks. Many wearers find the breathing resistance objectionable. Some wearers report suffering from mild hypoxia-induced fatigue. And as is the case with single-use masks with exhaust valves, if the wearer has a respiratory infection, this form vents pathogens into breathable airspace.

Secondly, this form is much heavier; generally weighing 250-350 gm compared to 4-5 gm single-use forms. Accordingly, sturdier bands and higher elastic tension is needed to hold the facepiece and attached filter media on the face and to maintain a reliable seal. For many people, this form is more unpleasant to wear, more cumbersome than the disposable form.

Thirdly, the skin areas in contact with the non-porous resilient plastic edge easily become sweaty. High elastic pressure together with sweat, may for some people if worn for an hour or more, cause skin irritation, contact dermatitis and pressure urticaria.

Furthermore, most consumers regard this form as industrial equipment, inappropriate, aesthetically unattractive, too encumbering; not worn outside the industrial context.

Finally, because of the deficiencies of both these forms, the greater majority of people, particularly in western cultures, dislike wearing them and generally don't wear them unless they feel the pathogen threat is extraordinarily high, or unless required to do so by an employer or a superior authority.

Positive Pressure Respirators

Known, positive-pressure respirators provide filtered air under low pressure to the mask. The mild pressure within the mask cavity prevents ingress of unfiltered air. Most have transparent face shields to protect the eyes. However, they are much more costly than the previous forms discussed, very cumbersome and unsuitable for everyday use by the public. This form is taught by Klockseth et al., U.S. Pat. No. 5,950,621A (1999).

Electronic Forms

Electronic PPE devices treat air before delivering the treated air to the wearer's nose and mouth region. An example of this form is disclosed in Courtney, Patent Application No. GB2575812 (2020). The specification teaches head-mounted music headphones with air filtering, wherein a high-speed impeller draws air through filter media and conveys filtered air through piping to an outlet near the mouth and nose.

This form has several disadvantages. Firstly, the head of the wearer is encumbered. Secondly, the filter media may become contaminated. If a high pathogen load builds in the media, and the media is not replaced, pathogens may be drawn through the media into inspired air.

Thirdly, high-speed air impellers cannot be noiseless, especially when mounted inside cups directly over each ear. Electronic motor bearings, turbulence created at the edges of impeller blades, and airflow through ducting and grills generate audible sound which many people may find unpleasant or irritating. Further, the hearing of a wearer may be adversely affected which may be dangerous in certain environments.

Another of these electronic forms, disclosed in Wei et al., U.S. Patent No. US20050284470A1 (2005) teaches a device comprising a micro-air processor mechanism and an airflow control mechanism. The micro-processor draws in and then processes the air by either filtering, heating, treating it with a disinfectant, pharmaceutical or air freshener. The air is then delivered by a fan to the wearer to be breathed.

Several disadvantages are inherent in the Wei device. Firstly, drawing air into the micro-processor requires a fan. It is known that airborne particulates build up on the surfaces of fans, air ducting and fan grills. Regular inspection and cleaning are required lest pollutants and pathogens build up in the micro-processor assembly. It is inevitable that some of the pollutants and pathogens periodically detach and are swept along in the air delivered to the wearer. Further, variants incorporating filter media require frequent replacement of the media to ensure the filter media does not build up pollution particles and pathogens and that some are stripped off by airflow and travel in the air delivered to the wearer.

In yet another form of electronic personal protection device taught by Weiberg, U.S. Pat. No. 5,484,472 (1996) a small air purifier is provided. This form provides an air chamber wherein a high voltage corona discharge is generated to ionize and ozonate the air. Air is drawn into the chamber by a fan or other means passed through the corona discharge and moved onward to the wearer to be breathed. Some forms of the device include filter media to remove toxic ozone generated by the device before delivering the treated air to the wearer.

The disadvantages of this form are numerous. The electrode and electrified grill used to generate the corona discharge may produce sparks that may ignite volatile vapour. The device may ignite gasoline vapour at an automobile filling station, or gas, paint solvents and the like in the home, public and workplaces. Further, ozone generated by the device is a known respiratory tract irritant. Although some forms of the device may include a filter to remove the ozone, leakage of ozone would adversely affect the wearer; particularly if the wearer is suffering any form of respiratory illness. Furthermore, as with all other air filtering devices, infrequent replacement of the filter media may result in contamination of the treated air delivered to the wearer.

All existing forms have significant disadvantages. Mask forms are either disposable, do not seal well, are uncomfortable, encumbering, restrict airflow, become a source of air contaminants, or require filter changes so as not to become a source of contaminants. Disposable forms are wasteful of finite natural resources, require bio-hazard disposal methods and are a new source of pollution. Reusable forms require careful monitoring of filter media and frequent replacement lest it become a source of air contamination, restrict airflow, are face and head encumbering, heavy, unpleasant to wear.

Electronic forms, unless often cleaned may also become a source of contaminants and pathogens. Unless carefully monitored, maintained and cleaned, the air these devices treat and deliver to the wearer may contain pollutants and pathogens released from a build-up of toxins and pathogens in the device's filter material, within air chambers, on fans and air conveyance means.

Furthermore, particularly in western cultures, the majority of people avoid wearing head and face encumbering masks and aesthetically unappealing devices of any form.

Consequently, unless required to do so by employers or government authorities most people in the USA and other western world cultures avoid wearing existing forms of PPE. Evidently, most prefer to risk infection rather than suffer their many disadvantages. Billions of people, therefore, remain at risk of infection from SARS-CoV-2 and all other airborne pathogens. Hundreds of millions suffer serious infection each year. Each year about 5 million people, young and old, suffer and die of an airborne transmitted respiratory disease.

A reference herein to a patent document or any other matter identified as prior art is not to be taken as an admission that the document or other matter was known or that the information it contains was part of the common general knowledge as at the priority date of any of the claims.

### BRIEF DESCRIPTION OF THE PRESENT INVENTION

Embodiments of the present invention can negate or ameliorate some or all of the many disadvantages of existing forms of PPE. Further, that the device may be small allows for a discrete, aesthetically attractive design to harmonize with people's workwear, casual wear and formal wear. Smallness and appealing aesthetics are generally considered a subordinate cosmetic to functionality, however, because billions of people prefer to risk infection rather than be encumbered by an existing form of PPE, smallness and appealing aesthetic are major functional benefits of the present invention—as it will certainly encourage mass usage of an effective PPE to prevent mass suffering and prevent many millions of deaths.

To date, despite major advances in medical knowledge and technology, the global scale of infection, suffering and deaths from airborne transmission of respiratory diseases remains staggering. Evidently, current forms of PPE fail to protect billions of people from infection. Every year many billions of people inspire airborne pathogens. Hundreds of millions suffer debilitating symptoms. Tens of millions of cases develop into a life-threatening disease. Each year some 5 million people, young and old, succumbed to a preventable, airborne respiratory disease, suffer and die.

Even with the danger of highly infectious SARS-CoV-2 pathogen and lethal disease COVID-19 daily in the international news, reported infection rates and the death toll rising, billions of people risk infection rather than suffer the disadvantages of existing encumbering, aesthetically unattractive personal protection devices. Free of all the disadvantages identified with existing devices, many of those billions may adopt usage of the present invention, and in doing so will protect themselves and those in their community, from the spread of SARS-CoV-2 and all other airborne pathogens.

It can be expected, that if many in the community use the present invention, the normal high pathogen load in that community's air, will be greatly reduced—thus reducing the incidence of clinical infection and disease for all in the community.

The present invention does not filter the wearer's air, nor does it treat and deliver air to the wearer. The present invention uses sensors to assess the likelihood of the presence of various forms of pathogens in the wearer's airspace and shoots biocide fluid droplets at target pathogens and pathogen containing respiratory droplets.

The shot biocide fluid droplets are sized and shot at a speed such that at least a portion impact pathogens and pathogen containing respiratory mucus droplets in their shot path, and so that a portion that does not strike a said target is slowed by drag and become suspended in the wearer's breathed airspace.

In complex ways described by the Stokes and Reynolds formula factoring variables including droplet mass, aerodynamic shape, velocity, air turbulence, air temperature, relative humidity, droplet electrostatic charge etc., suspended droplets enjoin the mildly turbulent air and intercept pathogens and pathogen containing respiratory droplets.

At impact and interception, biocide contacts pathogens and destroys or inactivates them so they are not infectious. At impact and interception of a biocide fluid droplet with a respiratory droplet containing pathogens, the droplets coalesce to form one larger droplet. Two protective effects immediately occur.

Firstly, coalescence results in diffusion of the biocide throughout the new droplet and destruction or inactivation of all or at least most contained pathogens. Secondly, larger in diameter and mass, the coalesced droplet is made somewhat safer, because larger droplets settle out of the airstream more quickly, and if inhaled their greater inertia causes them to leave airstream at air direction change to impact and become trapped in the respiratory tract mucus, generally in the upper respiratory tract; rather than remain in the airstream to travel deep into the lower respiratory tract.

It is known that upper respiratory tract depositions may be removed by excretions or by the Mucociliary Escalator, and any infection that may occur may be limited to less severe upper respiratory tract infection. Infections of the lower respiratory tract, especially the alveoli, which are not protected by mucus, are known to require a lower pathogen load, result in more severe symptoms, more rapid progression of disease and more deaths.

The present invention shoots biocide fluid droplets to effect protection in at least one of eight ways:
1) Directly hitting pathogens and pathogen containing droplets in the wearer's airspace.
2) Directly hitting pathogens that have deposited on the mucosa of the wearer's eyes, nose and mouth.
3) Intercepting of pathogens that have deposited on the mucosa of the wearer's eyes, nose and mouth.
4) Intercepting pathogens and pathogen containing droplets in the wearer's airspace.
5) Intercepting pathogens and pathogen containing droplets in the airstream in the wearer's respiratory system.
6) Deposition on pathogens that have deposited on surfaces within the respiratory system.
7) Prewetting of the mucosa of the wearer's eyes, nose and mouth by deposition of shot biocide fluid droplets, so that pathogens that subsequently deposit thereon are inactivated.
8) Prewetting of surfaces of the wearer's respiratory system by deposition of shot biocide fluid droplets, so that pathogens that subsequently deposit thereon are inactivated.

Broadly, the preferred embodiments of the present invention protect the wearer from pathogens while providing eight major advantages:
1) The wearer's face is left free, mask-less, apparatus-less, unencumbered.
2) No filter media is used, which negates the risk of air contamination.
3) The device cannot become contaminated because air is not passed through it, not treated and delivered to the wearer.
4) Having no filter element, no fan, no associated air ducts and grills the present invention may be small and lightweight.
5) Having no fan, no blower, not moving air, the device may be silent.
6) Having no electrical components with a large power draw, the device may be powered by a small battery.
7) Uses minute amounts of biocide in a plurality of micro-size droplets the biocide fluid reservoir may be small.
8) The device may be small, unobtrusive.

Shot size and speed is a critical factor. Shot droplets rapidly equilibrate. Depending on the temperature and relative humidity, water may evaporate off its surface and reduce the droplet to half its diameter in <1 sec. Reduced in mass the shot is rapidly slowed by air drag. Shot size and speed are optimized so the shot droplet may carry at speed through to at least the centre of said airspace, before losing forward momentum and becoming suspended in turbulent airflow, whereafter it may intercept pathogen containing droplets in that air.

Forms of the present invention may shoot droplets of various size at various shot speeds. A shot size 5-10 μm diameter, and a shot speed of 0.5-2.0 meters per second is preferred. This shot size and speed is advantageous for reasons which include:
1) At maximum expected equilibration to 50% diameter, 12.5% mass in <1 sec, a shot may be expected to carry at least 150 mm, to strike airborne pathogens and respiratory droplets along its path, before being slowed by air drag and becoming air suspended if a pathogen or respiratory droplet is not hit.
2) An equilibrated shot of ≈0.5 μm may be inspired, remain in the airstream and may deposit in either the upper or lower respiratory tract.
3) Each 1.0 ml of biocide fluid may produce >19 billion shots.
4) Each 100-millisecond burst may shoot >1 million shots.
5) Each shot may contain a minute volume of biocide.
6) Biocide ppm concentration may be such that it inactivates pathogens to log-4 (99.99%) in less than 60 seconds, yet 8-hour total aerosol exposure may be virtually non-toxic to humans.

Preferably, the present invention uses low cost, lightweight piezoelectric vibrating mesh technology (VMT) to simultaneously generate and shoot <10 μm biocide fluid droplets at target pathogens in the wearer's said airspace, in inspired air and one the wearer's mucosa. Developed in 1983, VMT elements of various meshes and frequencies are commonly available. VMT elements of various size and frequency may be used in various forms of the invention.

Preferably, the present invention comprises a biocide fluid reservoir, at least one said VMT, an electronic circuit and related components (hereafter ‘electronics’), and means of removably locating the device to the chest area of the wearer which may include at least one of a) a magnet and a co-attractive element, (b) clothing pin, (c) a resilient clip, (d) a hanging hook, (e) a necklace, (f) a neck string.

Preferably said biocide fluid is conveyed to the underside of at least one VMT element by at least one of a) wicking element, (b) a pump, (c) gravity.

In a simple form, the invention may be manually controlled to intermittently shoot microsecond or longer bursts of biocide fluid droplets over a protection period.

In a sophisticated form, the invention may be used in conjunction with a mobile device and a central processor unit (CPU) and have various sensors which may include a biocide fluid level sensor, camera, gesture sensor, barometric pressure-temperature-humidity, and a Bluetooth device proximity scanner.

The device may also include an accelerometer-gyroscope, microphone, and a software application (app) on a smartphone, tablet or the like so that the wearer has many functional, status and notification options effected through said mobile device and app. Further, said, Smartphone may have GPS capability, which GPS data may be accessed by the device so that location of the wearer of the device may also be known and factored.

In a more sophisticated form, an element of artificial software (AI) is included, to enable AI to autonomously, intelligently operate the device to provide the wearer optimum protection in changing circumstances, while making the most effective use of biocide fluid. Said AI may be adapted to learn the wearer's behaviour, anticipate the wearer's behaviour in various social settings and environments and take pre-emptive protective action,

It is to be noted that due to the wetting action of prior shots of biocide fluid, a continuous emission of biocide fluid droplets is not necessary and would be wasteful. Continuous emission may consume a large quantity of biocide fluid, likely a litre or more of solution over 8 hours or more of usage. Interfacing with said components and sensors, AI is used to continuously determine changing pathogen risk level. Informed by said sensors, AI may take decisions too variable to detail here. However, several examples are here presented:
a) Based on data from GPS, camera, microphone, barometric pressure, temperature and humidity sensors, and Bluetooth device proximity scanner, AI may determine the probability of air pathogen load and increase or decrease shots of biocide fluid droplets, to optimize protection, biocide fluid and power usage accordingly.
b) Informed by the accelerometer and gyroscope data, together with computed risk level, AI may choose to deliver droplets bursts between the wearer's exhalation and inhalation, to maximize dwell time of the droplets in said airspace.
c) Sensors may detect a person within the intimate personal space (within about 1 metre of the wearer), and AI may determine risk is minimal and may pause droplet shots to allow intimacy.

Preferably all forms use the smallest practicable quantity of biocide fluid; preferably <20 ml over an 8-hour protection period. Preferably said biocide fluid is a low cost, low toxicity to humans OTC biocide agent miscible in water or dispersible in water by use of safe, common low-cost solvents or emulsifying additives. Biocidal agents may include saline, alcohol, various diluted solubilized essential oils and any biocide agents safe for use by humans.

Preferred agents may be any suitable biocide including but not limited to Triethylene glycol, H2O2, Cinnamomum zeylanicum, Propylene Glycol; used in small quantity (preferably less than 1 ml per 8 hours), all diluted to levels known to be safe for human inhalation by aerosol.

Preferably, said reservoir is formed from two plastic injection moulded parts ultrasonically welded together to form a biocide fluid container which also functions as the device chassis; which chassis has front, back, top and bottom walls.

Preferably the reservoir is refilled through an orifice having an air venting and fluid shut of valve, and the reservoir refilled by use of a plastic syringe or a disposable plastic ampoule having a tapered Luer slip like nozzle. Preferably, said top wall has at least one reservoir hole therein, at least one VMT seated thereon.

Preferably said front, top and bottom walls have an electronics circuit board with various electronic components mounted or attached thereto. Preferably said device chassis has a front cover, covering and protecting the circuit board and electronics affixed thereto.

Preferably said chassis cover has an aperture in a front face reciprocal to a lens of a camera mounted on said electronics circuit board, and a translucent area to allow the display of LEDs.

Preferably said chassis cover is adapted to accept releasable attachment of various designs of front covers which are made available to the user. Preferably said front covers are formed in transparent, coloured, painted, printed or decorated material. Wearers may match front covers designs to apparel worn and or attach any available front covers design option the wearer finds appropriate or appealing.

Preferably, some forms of the present invention may impart an electrostatic charge to the biocide fluid droplets as they are shot into the wearer's airspace, to add electrostatic attraction to inertial impaction and interception, to ensure the highest level of collision efficiency. Depending on the expected net charge of pathogen containing droplets emitted by a person suffering a particular form of respiratory infection, the electrostatic charge given the biocide fluid droplet may be a positive or a negative charge. Preferably, at least a portion of the said droplets generated by said VTM may be caused to carry a positive electrical charge, because medical research presently evidences that respiratory mucus, a major component of respiratory droplets, often carries a net negative charge. Alternatively, the device may impart a negative electrical charge to shot biocide fluid droplets to increase the interception rate of pathogens and pathogen containing droplets known to be carrying a net positive charge.

Accordingly, there is provided a wearable personal protection device for protecting a wearer against airborne pathogens comprising;

a battery-powered electronic circuit and components for controllable operation of at least one piezoelectric vibrating mesh transducer which is configured to intermittently simultaneously generate and shoot droplets of a biocide fluid into the wearer's proximal airspace to contact and inactivate at least some of the pathogens which may be present;

a reservoir for containing the biocide fluid therein; and

a fluid delivery apparatus for conveying said biocide fluid to an uptake location of said piezoelectric vibrating mesh transducer.

In especially preferred embodiments of the invention the fluid delivery apparatus is at least one of a (a) wicking element, (b) pump, (c) gravity mechanism for conveying said biocide fluid to an uptake location of said piezoelectric vibrating mesh transducer.

In some preferred embodiments of the invention, the wearable personal protection device further comprises at least one of (a) a magnet and a co-attractive element, (b) a clothing pin, (c) a resilient clip, (d) a hanging hook, (e) a necklace, (f) a neck string, for removably locating said wearable personal protection device on the upper front region of a wearer.

In other preferred embodiments, the wearable personal protection device operates such that at least a portion of said biocide fluid droplets are shot into the wearer's airspace such that said droplets enjoin the wearer's inspired airstream to travel into the wearer's respiratory system to contact with and inactivate at least a portion of pathogens which may be in the said inspired air stream.

In other embodiments, the wearable personal protection device operates such that at least a portion of said biocide fluid droplets are shot at the mucosa of the wearer's face so at least some of said droplets may strike and inactivate at least a portion of said pathogens which may be thereon.

In additional preferred embodiments, at least a portion of said biocide fluid droplets enter the wearer's respiratory system and deposit on surfaces therein to inactivate at least a portion of said pathogens that may have deposited thereon.

In further preferred embodiments, at least a portion of said biocide fluid droplets are shot at the mucosa of the wearer's face to wet said mucosa with said biocide fluid to inactivate at least a portion of pathogens which may subsequently deposit thereon.

In still other preferred embodiments at least a portion of said biocide fluid droplets enjoin the wearer's inspired air and enter the wearer's respiratory system to wet at least a portion of the surfaces of the wearer's respiratory system to inactivate at least a portion of pathogens which may subsequently deposit thereon.

Preferably, at least a portion of said biocide fluid droplets are imparted with either (a) a positive electrical charge, or (b) a negative electrical charge, to increase collision efficiency with neutral or oppositely charged droplets and particles.

In especially preferred embodiments of the invention, the shot biocide fluid droplets contain at least one of (a) Triethylene glycol, (b) H2O2, (c) Propylene glycol (d) an aromatic oil; diluted to a level known to be safe for use by humans while remaining efficacious for inactivation of at least some pathogens contacted.

Preferably the wearable personal protection device of the present invention includes a central processing unit chip (CPU); a wireless receiver and transmitter component, which communicates with and detects the proximity of wireless devices; a software application (app) on a mobile device, wherein the electronic circuit co-acts with said CPU, a wireless receiver and transmitter to allow at least one of (a) setting, (b) control of, (c) viewing of device status, (d) notifications and reports; with said app on said mobile device.

In other preferred embodiments, the wearable personal protection device includes at least one of (a) a pair of electrodes positioned inside the said reservoir, which electrode pair is adapted to sense biocide fluid level in the reservoir and communicate same to said CPU and said app for display on said mobile device, (b) a transparent area of said biocide fluid reservoir so that the biocide fluid level is visible.

Preferably, the wearable personal protection device includes a haptic feedback component which is used to alert the wearer, via different pulses, of at least one of (a) biocide fluid level, (b) battery charge level, (c) estimated pathogen levels, (d) various statuses of the device (e) likely presence of specific pathogens and irritants or a class of pathogen or irritant

In some embodiments the wearable personal protection device includes a gyroscope-accelerometer sensitive to the wearer's chest movements and adapted to enable synchronization of bursts of said biocide fluid droplets from said piezoelectric vibrating mesh transducer with at least one of (a) the wearer's exhalation, (b) wearer's inhalation, (c) the pause between the wearer's inhalation and exhalation. Synchronization with the wearer's exhalation gives protective priority to others in proximity to the wearer. Conversely, synchronization with the wearer's inhalation gives protective priority to the wearer. Synchronization with pauses in the wearer's inhalation and exhalation is neutral.

In especially preferred embodiments the wearable personal protection device includes artificial intelligence software (AI) which uses data from said electronic components to autonomously operate the said wearable personal protection device with the goals of customizing protection to the physiology, breathing rate, tidal volume, and behaviour of the wearer and usage of the minimum quantity of biocide fluid needed to inactivate a majority of pathogens computed to be likely present in the wearer's said proximal breathing air space.

Preferably the wearable personal protection device includes a barometric pressure, temperature and humidity sensor, which supplies data to at least one of said (a) CPU, (b) app, (c) AI software, which uses the data to determine the probability of pathogen presence in said proximal breathing air space, and to determine the likely viability of a specific pathogen or type, said viability being known to be dependent on barometric pressure, air temperature and humidity. said air barometric pressure, temperatures and humidity levels relative to at least some of known pathogens being made assessable by at least one of said components (a), (b) and (c).

In some preferred embodiments the wearable personal protection device includes a camera component, to detect proximal people; a microphone, to allow the said wearable device to respond to the sound of coughs, sneezes and speech of said proximal people, which sounds are known to produce respective levels of emissions of respiratory mucous droplets which are a source of pathogens; said components, together with data from other said sensors support computation of risk level and computation of an effective immediate protective response by said wearable personal protection device.

In some preferred embodiments the wearable personal protection device incorporates an app which is used on a mobile device which uses a least one of (a) a global position system component (GPS), (b) mobile device location by triangulation of cell tower locations, (c) wi-fi location positioning, utilized by said the app so the wearer's geographical location may be computed, and together with data from said other sensors including at least one of (a) camera, (b) a gesture sensor (c) barometric pressure sensor, (d) humidity and temperature sensor, (e) wireless transmitter and receiver, (f) an accelerometer-gyroscope (g) a microphone, whereby the pathogen threat level may be computed and communicated to the wearer by at least one of (i) haptic feedback signal on the said wearable personal protection device, (ii) app initiated sound emitted from the said wearable device and or from the said mobile device, (iii) imaged displayed by said app on said mobile device (iv) text displayed by said app on said mobile device (v) an audio speech virtual assistant on said mobile device; and the wearable device may also automatically generate biocide fluid droplets commensurate with said computed threat level.

Preferably, data recorded by the wearable device is used by at least one of (a), said app (b), said AI software to prepare periodic threat and threat mitigation action reports, which the wearer may access and view displayed by the said app on said mobile device;

said prepared reports enable the wearer to learn of high threat times, locations and situations to allow for proactive minimization of exposure to high pathogen loads.

In some preferred embodiments the biocide fluid reservoir includes a biocide fluid filling port comprising; a hollow boss integral with a wall of said biocide fluid reservoir; a hollow form with an open end shaped to accept a Luer-slip type tapered nozzle therein and having a closed end, said hollow form being slidably mounted within said hollow boss; further comprising; a coil spring captured between an annular ring at said open end of said slidably mounted hollow form and an opposite annular ring near the end of said hollow boss; said slidably mounted hollow form and said spring is captured within said hollow boss by an O-ring in an annular trench; said slidably mounted form further having at least one inwardly directed hole in its wall to allow biocide fluid ingress to said reservoir, and at least one outwardly directed hole to allow air venting from said reservoir during biocide fluid ingress when the spring is fully compressed by forceful insertion of said filling nozzle; closure of said ingress hole and said air venting hole is effected when the Luer-slip type tapered nozzle is removed and said spring resiliently returns said slidably mounted hollow boss to a rest position; and whereby said o-ring is simultaneously forcedly re-seated by said spring in an internal annular recess at the open end of said hollow boss.

In especially preferred embodiments the biocide fluid reservoir includes a biocide fluid filling port comprising: a tapered orifice having at the narrow end a fluid egress prevention valve; said tapered orifice sized to accept a reciprocal tapered nozzle fitted to a separate biocide fluid refill pack; which refill pack co-acts with said tapered orifice to effect fast, easy refill of said personal protection device.

Preferably, the biocide fluid reservoir includes a biocide fluid filling port comprising: a hollow boss integral with a wall of said biocide fluid reservoir; a hollow form with an open end shaped to accept a nozzle therein and having a closed end, said hollow form being slidably mounted within said hollow boss; further comprising a coil spring captured between an annular ring at said open end of said slidably mounted hollow form and an opposite annular ring near the end of said hollow boss; said slidably mounted hollow form and said spring is captured within said hollow boss by an O-ring in an annular trench; said slidably mounted form further having at least one inward hole to allow biocide fluid ingress to said reservoir, and at least one outward hole to allow air venting from said reservoir during biocide fluid ingress when the spring is fully compressed by forceful insertion of said filling nozzle; closure of said ingress hole and said air venting home is effected when Luer-slip type tapered nozzle is removed and said spring resiliently returns said slidably mounted hollow boss to a rest position; and whereby said o-ring is simultaneously forcedly re-seated by said spring in an internal annular recess at the open end of said hollow boss.

Preferably, the wearable personal protection device includes at least one ring-shaped electrode positioned above said piezoelectric vibrating mesh transducer; a ground electrode in said biocide fluid reservoir; said ring-shaped electrode and said ground electrode adapted to impart a positive electrical charge to at least some of the biocide fluid droplets generated and propelled by said piezoelectric vibrating mesh transducer; to increase collision rate of said droplets with at least one of (a) respiratory mucus droplets containing pathogens, (b) pathogens not contained in mucus droplets; which mucus droplets and pathogens are expected to be predominantly negatively charged.

In preferred embodiments the wearable personal protection device further includes; at least one ring-shaped electrode positioned above said piezoelectric vibrating mesh transducer; a ground electrode in said biocide fluid; said ring-shaped electrode and said ground electrode adapted to impart a negative electrical charge to at least some of the biocide fluid droplets generated and propelled by said piezoelectric vibrating mesh transducer; to increase collision rate of said droplets with at least one of (a) respiratory mucus droplets containing pathogens, (b) pathogens not contained in mucus droplets; which mucus droplets and pathogens are expected to be predominantly positively charged.

Preferably, the app uses said mobile device to wirelessly access a database that supplies information relative to the locations of known sources of pathogens and uses said information together with other data to effectively operate the said wearable personal protection device.

In some preferred embodiments, the wearable personal protection device further comprises at least one motion sensor component, so that on and off and various functions of the device may be controlled by hand gestures of the wearer communicating with said motion sensor.

In other preferred embodiments, the wearable personal protection device further comprises at least one microphone component and an audio processor for sound and or speech recognition, which enables at least one of (a) on and off control, (b) various functions, by the wearer's utterances.

Preferably the wearable personal protection device has a battery charging port.

In especially preferred embodiments the wearable personal protection device further comprising a releasable front cover, comprising; at least one aperture in a top wall and at least one aperture or indent in a bottom wall, which apertures or indents are reciprocal to short protrusions in the body of said personal wearable device; said top and bottom walls being formed of sufficiently resilient material to allow said walls to flex outward from said protrusions and flex back to releasably capture said protrusions in said apertures or indents when said front cover is forced against said protrusions; whereby the wearer may release the cover and replace it with another or differently designed cover.

In other preferred embodiments the front cover of the wearable personal protection device is provided as a least one of (a) transparent plastic moulding, b) a transparent plastic moulding having an inner surface printed with a colour and or image, (b) a plastic moulding variously coloured, printed or decorated; whereby the wearer may select from a multiplicity of front cover designs provided and attach personally, preferred front cover designs to express their individuality, artistic preference, or alter the appearance of the said personal wearable device relative to clothing being worn. Said front cover may also be of any shape to change the outward form of the device.

In especially preferred embodiments of the invention, the biocide fluid reservoir is formed of moulded plastic material.

In other preferred embodiments, the biocide fluid reservoir is formed of moulded plastic material and provides a tapered biocide fluid filling port with integral fluid ingress and air egress valve, sized to accept a standard tapered medical syringe nozzle.

In especially preferred embodiments of the invention at least one piezoelectric vibrating mesh transducer controllably simultaneously generates and shoots biocide fluid containing droplets upwardly toward the airspace forward of the wearer's face.

Preferably, the wearable personal protection device further comprises a replaceable outer cover of at least one of (a) a transparent material, (b) a different shape, (c) a different pattern thereon, (d) a translucent material, (f) printed inside or outside with text, image or pattern, or a combination thereof, (g) made of a different material, (h) fabric-covered, (i) decorated; whereby the wearer may express their individuality, artistic preference, or alter the appearance of the said personal wearable device relative to clothing being worn or personal preference.

There is also provided a method of personal protection from pathogens, comprising the steps of:

(i) providing an assembly comprising a biocide fluid in a reservoir; a battery-powered electronic circuit and at least one electronically powered piezoelectric vibrating mesh transducer to shoot biocide fluid droplets;

(ii) providing at least one of (a) a wicking element, (b) a pump, (c) gravity to convey said biocide fluid to the underside of said piezoelectric vibrating mesh transducer;

(iii) providing at least one of (a) magnet, (b) clothing pin, (c) a resilient clip, (d) a hanging hook, (e) a necklace, (f) a neck string for removably locating said wearable personal protection device on the upper front area of a wearer;

(iv) orientating said assembly such that said assembly when activated controllably shoots said biocide fluid droplets into the proximal breathing air space and face of the wearer; whereby said biocide fluid containing droplets may contact with and inactivate pathogens in at least one of the following manners: (a) collision within said breathing air space, (b) interception and by Brownian motion and or electrostatic attraction in the wearer's inspired air, (b) deposition on at least a portion of surfaces of the wearer's respiratory system, (c) deposition on at least a portion of the mucosa of the wearer's face.

### BRIEF DESCRIPTION OF DRAWINGS

Preferred embodiments of the present inventions will now be described with reference to the accompanying drawings wherein:

FIG. 1 is a perspective view of a first preferred embodiment of a wearable personal protection device and a smartphone with related app screen shown.

FIG. 2 is a perspective view with a part of a wearer's shirt cutaway, showing how the wearable personal protection device of FIG. 1 is removably located magnetically to a thickness of clothing.

FIG. 3 is a sketch showing the wearable personal protection device of FIG. 1 removably located on clothing on the chest area of a woman and the shoulder of a man, shooting biocide fluid droplets into the airspace forward of the face of the wearer.

FIG. 4 is a perspective sketch of a human head showing a dotted area of a volume contiguous with the wearer's face extending back a little past the ear canal, which volume is referred to in this specification and the claims as the wearer's airspace.

FIG. 5 is a perspective sketch of a human upper body showing a dotted area where the wearable personal device may be removably located.

FIG. 6 is a view of the wearable personal protection device FIGS. 1, 2 and 3, with a cutaway showing the PCB and magnetic removable location means.

FIG. 7 is an exploded perspective of the chassis of the wearable personal protection device of FIGS. 1, 2, 3, and 6 showing attached components, circuit board and cover.

FIG. 8 is an exploded perspective of the wearable personal protection device of FIGS. 1, 2, 3, 6 and 7 showing the assembled body and the removable front cover.

FIG. 9 shows a magnetic USB charging cable and charging port of the device.

FIG. 10 shows orthographic views (front, top, side, bottom and back) of the wearable personal protection device of 1, 2, 3, 5, 6, 7, 8 and 9.

FIG. 11 shows section views of the wearable personal protection device of FIG. 10 along section lines A-A, B-B.

FIG. 12 is a section view of the wearable personal protection device of FIG. 10 along section lines C-C, showing the biocide fluid filling port and air release valve.

FIG. 13 is a front view of an alternative filling port with an integral air vent and two section views at line A-A showing i) the valve closed and ii) the valve open during filling.

FIG. 14 shows the biocide fluid reservoir of the device being refilled with a standard plastic syringe having a Luer-slip type tapered nozzle.

FIG. 15 shows the biocide fluid reservoir of the device being refilled with a sachet having a Luer-slip type tapered nozzle.

FIG. 16 shows the biocide fluid reservoir of the device being refilled with biocide fluid packaged in a plastic ampoule having a shortened Luer-slip type tapered nozzle.

FIG. 17 is a perspective view of a second preferred embodiment of a wearable personal protection device, using a piezoelectric vibrating mesh transducer, supplied biocide fluid by gravity.

FIG. 18 is a front view and a section view of the device of FIG. 17.

FIGS. 19 to 23 are perspective sketches of preferred means of attaching the personal protection device to the clothing of the wearer, additional to the first preferred magnetic attachment means shown of removably locating the wearable personal protection device on the front area of a wearer: FIG. 19 shows a clothespin, FIG. 20 shows a resilient clip, FIG. 21 shows a hook, FIG. 22 shows a neck-chain, FIG. 23 shows a necklace.

FIG. 24 is a third preferred embodiment of a wearable personal protection device of the present 45 invention wherein the device is generally circular in form.

### DETAILED DESCRIPTION OF PREFERRED EMBODIMENTS

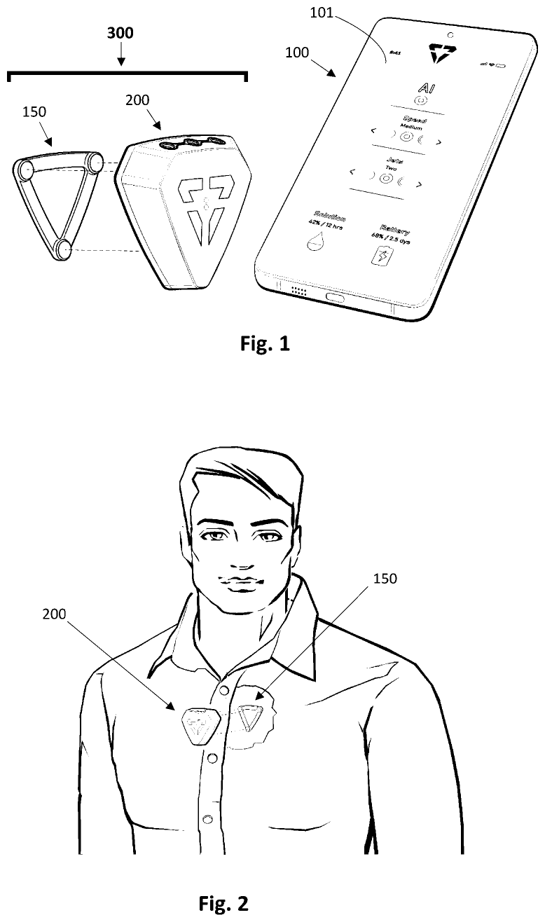

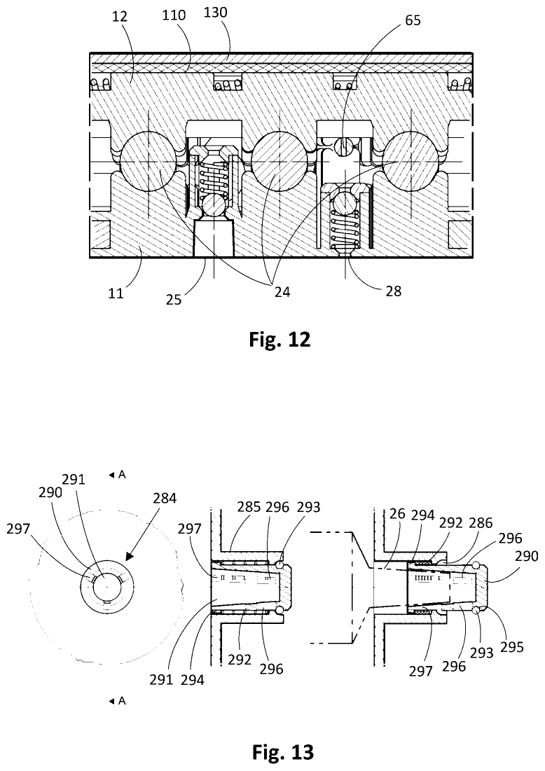

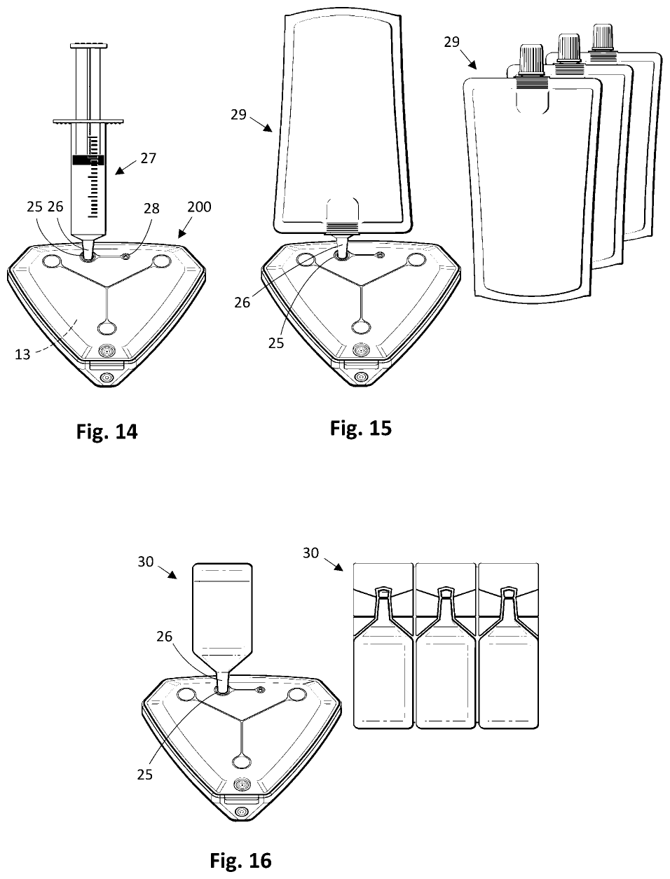

The base concept of the present invention allows for many embodiments. With reference to FIGS. 1 to 24, three preferred embodiments are herein presented:

### A First Preferred Embodiment

FIG. 1 shows a first embodiment of a wearable personal protection device 300 comprising; a device body 200, a mobile device 100 with a software application (app) 101, and a separate mounting element 150.

As best shown in FIG. 7, said device body 200 comprises; a chassis-reservoir assembly 10, an electronics assembly 60, a chassis-reservoir assembly cover 110, a releasable front cover 130.

Said device body interfaces with said mobile device software application (app) 101 which co-acts with an artificial intelligence software application (AI) in a central processor unit (CPU) 62 component of said electronics assembly 60.

Said mobile device app 101, may include at least one of (a) selection options of device function modes, (b) wearer notification: visual and or audio of biocide fluid level, battery level, current threat level for various pathogens.

Said AI uses an algorithm to react to real-time data from sensor components of said electronics assembly 60 to determine the most effective action to protect the wearer and or proximal others from a probable pathogen load in any environment. The most effective action determined by AI may include at least one of (a) haptic feedback signalling the wearer to leave an area which AI has determined likely has a dangerously high level of pathogens, (b) increased biocide fluid shot time length and frequency of generation of shots, (c) reduced shot time length and frequency of shots of biocide fluid droplets, (d) synchronization of shots of biocide fluid droplets with the wearer's breath intake, (e) synchronization of shots of biocide fluid droplets with the wearer's exhalations when the user decides to protect others (a proximal person such as an elderly person for example) from the probability of pathogens emitted in the wearer's exhalations, (f) no action.

Further, said AI may be updated wirelessly from a remote server to periodically enhance AI effectiveness. Updates may include at least one of (a) edits to AI data-set re various pathogens, (b) data re new pathogens (c) enhanced AI algorithms.

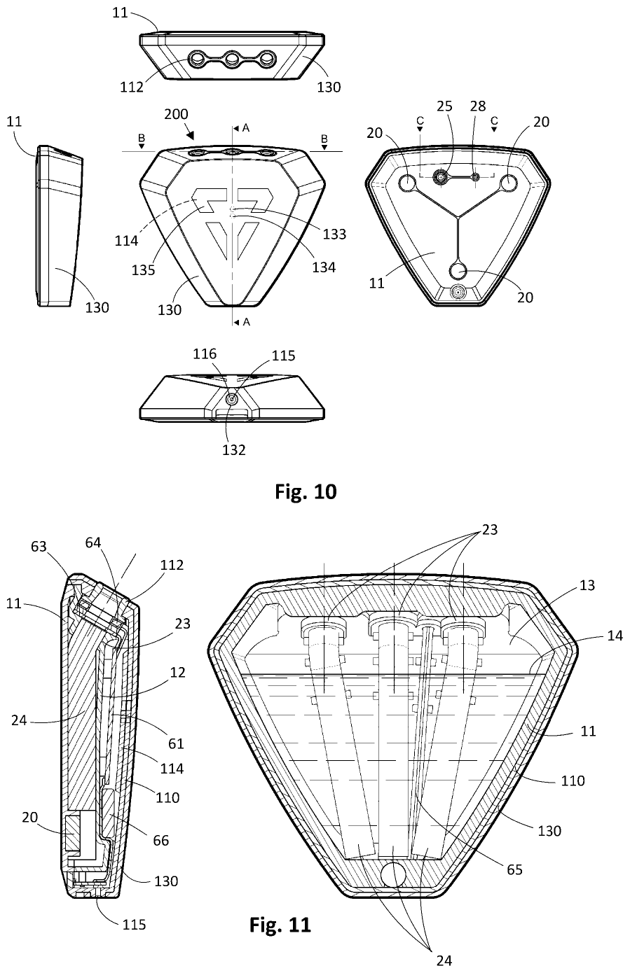

Said chassis-reservoir assembly 10 is formed by ultrasonic welding of chassis-reservoir back moulding 11, and chassis-reservoir front moulding 12 to provide a fluid reservoir 13 to contain a biocide fluid 14 as shown in FIG. 10.

Referring to FIG. 7, chassis-reservoir front moulding 12 provides mounting locations 16, 17, 18, 19 for said electronics assembly 60.

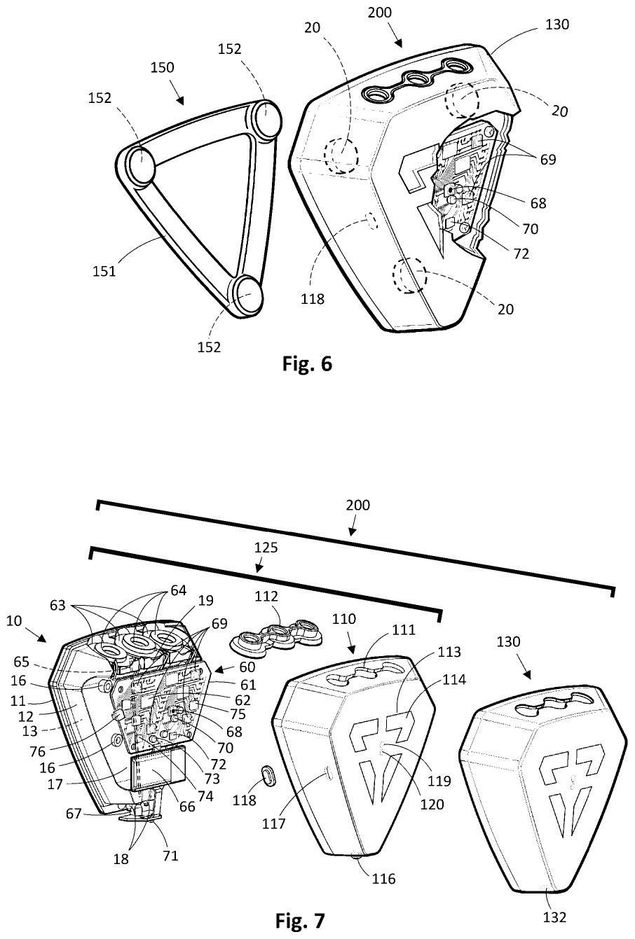

As best shown in FIGS. 6 and 10, said back moulding 11 has magnets 20 fixedly attached to co-act with magnets 152 of mounting element 150.

The device 300 includes an electronics assembly 60 that is contained within a chassis cover (an outer protective housing) 110 that is in electrical communication with one or more piezoelectric vibrating mesh transducers 63 to selectively control the release of biocide fluid 14.

As best shown in FIG. 7, said electronics assembly 60 comprises a PCB 61, three piezoelectric vibrating mesh transducers 63 which convert said biocide fluid 14 into ≈7 μm droplets 15 and shoot said droplets upwardly into the wearer's airspace shown in FIGS. 3 and 4. The electronics assembly 60 further comprises three electrode rings 64 and co-acting ground electrode 65, a battery 66, and a charging port 67. Components on the PCB include; a CPU 62 which CPU includes at least one of (a) Random-access memory (RAM) (b) integrated non-volatile storage, (c) separate non-volatile storage, a camera 68, four RGB LEDs 69, to indicate status, a gesture sensor 70, barometric pressure, humidity and temperature sensor 71, wireless transmitter and receiver 72, a haptic feedback element 73, an accelerometer-gyroscope 74, a microphone 75, an on-off/pairing button 76, and a piezoelectric pulse generator, high voltage generator, and charging circuit (not shown). It is to be noted that said wireless transmitter and receiver 72 may be utilized to also detect proximity and estimate the distance of Bluetooth enabled devices from the transmitter and receiver 72 to detect the likely proximity of people (those people having Bluetooth or like wireless technology enabled devices such as smartphones, computer tablets, smartwatches, fitness trackers and the like) from the wearer.

The present invention acquires GPS data from the wearer's mobile device (smartphone, tablet and the like). Said data informs the device's operating system of the wearer's location. The app and or AI software may determine whether the wearer is indoors, outdoors, in a specific public or private space, in a space with air known to have a high pathogen load (a hospital or a doctor's waiting room for example) or in proximity to persons known to have an infectious respiratory disease, etc. Depending upon the estimated risk level, the AI software may temporality cease, decrease, increase or otherwise optimize output of the protective biocide fluid droplets 15 generated by the wearable personal protection device 200.

The mobile device software app 101 can use at least one of (a) a global position system component (GPS), (b) mobile device location by triangulation of cell tower locations, (c) wi-fi location positioning, utilized by the app 101 so the wearer's geographical location may be computed, and together with data from other sensors including at least one of (a) camera, (b) a gesture sensor (c) barometric pressure sensor, (d) humidity and temperature sensor, (e) wireless transmitter and receiver, (f) an accelerometer-gyroscope (g) a microphone, the pathogen threat level may be computed and communicated to the wearer by at least one of (i) haptic feedback signal on the said wearable personal protection device, (ii) app initiated sound emitted from the said wearable device and or from the said mobile device, (iii) imaged displayed by said app on said mobile device (iv) text displayed by said app on said mobile device (v) an audio speech virtual assistant on said mobile device, and the personal protection device 300 may also automatically generate biocide fluid droplets commensurate with said computed threat level.

Data recorded by the personal protection device 300 is used by at least one of (a), said app 101 (b), said AI software to prepare periodic threat and threat mitigation action reports, which the wearer may view displayed by the said app 101 on said mobile device 100. The prepared reports enable the wearer to learn of high threat times, locations and situations to allow for proactive minimization of exposure to high pathogen loads.

The said three electrode rings 64 together with a co-acting ground electrode 65 are used to impart a positive electrical charge to the emitted (shot) droplets 15 to increase collision efficiency with pathogen carrying mucous droplets which researchers have recently discovered carry a net negative charge. Alternatively, the emitted biocide fluid droplets 15 may be imparted with a negative electrical charge according to the nature of the pathogen or pathogens being targeted.

Said gesture sensor 70 interfaced with the CPU 62 and AI software allows the wearer to control the device 200 with various hand gestures.

Said accelerometer-gyroscope 74 is sensitive to the wearer's chest movement and is used to monitor breathing rate; which enables the AI software, also informed by said other sensors, to synchronize the generation of droplets 15 with at least one of (a) breath intake, (b) breath exhalation, (c) the pause between breath intake and exhalation to vary and optimise protection.

Said microphone 75 with software enables said AI software program to react to the wearer's and other people's speech, coughing or sneezing caused emission of mucus droplets and to make decisions to operate the wearable personal protection device 200 accordingly.

As shown in FIGS. 6 and 7 said electronics assembly 60 is connected via said wireless transmitter-receiver 72 to said mobile device app 101, as depicted on a smartphone screen 100.

Said AI software program is interfaced with a mobile device app 101 compatible with Android, macOS, IOS and Microsoft Windows and like operating systems. As said, AI uses said CPU 62 to process all data provided by all said electronic components and sensors to autonomously operate the device 200; including operating said piezoelectric vibrating mesh transducers 63, to optimize burst time and rate of generation of biocide fluid droplets 15 in accord with real-time computed pathogen threat level. Preferably, burst time may be from 0.1 to 60 seconds or more.

Further, said AI informs the wearer of various conditions by operating at least one of (a) the haptic feedback element 73, (b) audio or display of said mobile device.

Referring again to said reservoir 13, as can be best seen in FIG. 10, the reservoir 13 comprises three holes 23 in the upper wall, three wicks 24 which feed the biocide fluid in the reservoir 13 to the underside of said three piezoelectric vibrating mesh transducers 63, said electrode 65 to ground and provide electrical resistance information to allow said CPU 62 to compute the level of biocide fluid and send same to said app 101, a filling port with a one-way valve 25 shaped to accept a tapered nozzle 26 on a syringe 27 or a soft sachet refill pack 29, or on a plastic ampoule refill 30, and a pressure release valve 28 to allow air to vent when filling the reservoir 13.

As best shown in FIGS. 6, 8 and 11 chassis cover 110 fixedly attaches to said chassis-reservoir assembly 10 encasing the electronics assembly 60 and forming the device main unit 125 (without releasable front cover 130).

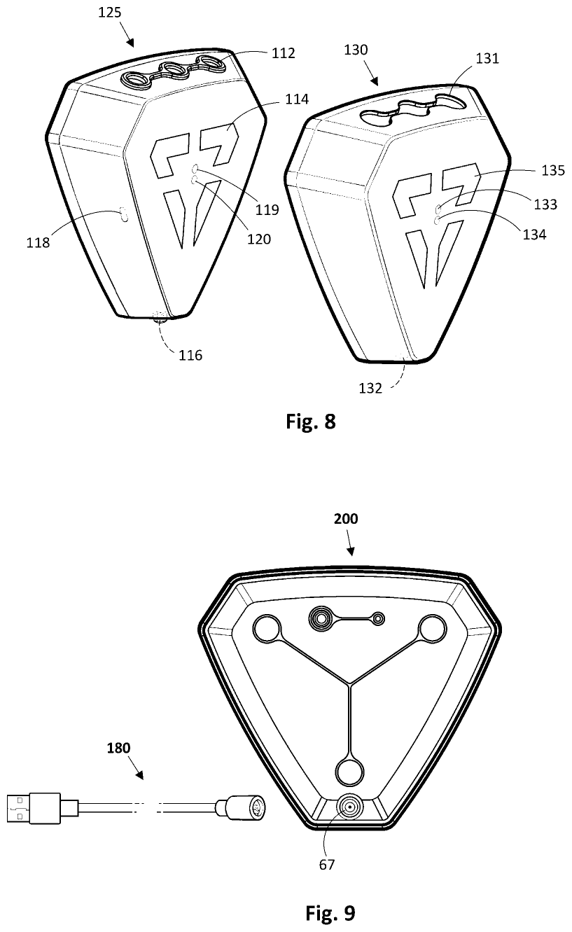

Said chassis cover is a plastic injection moulding 110 which comprises; an opening in the top face 111 45 which captures a bezel element 112 between said chassis-reservoir assembly 10, piezoelectric vibrating mesh transducers 63, said electrode rings 64 and said chassis cover moulding 110; said bezel element 112 best shown in FIG. 8 which protrudes at least part of the wall thickness of the releasable front cover 130 above the surface of said chassis cover moulding 110; and further comprises four openings 113 in the front face which accommodates a captured infill panel 114 moulded in translucent plastic material through which said RGB LEDs 69 may controllably emit light. Further, the chassis cover moulding 110 has a number of openings; an opening 66 coincident with the barometric pressure, humidity and temperature sensor 71, said opening 115 has a protrusion 116 which protrudes downwardly a least a portion of the wall thickness of the releasable front cover 130. Said chassis cover moulding 110 also has one small opening 117 in a side face which accommodates a plastic moulded button cap 118, which is reciprocal to said on-off/pairing button 76, and further has two openings 119, 120 on the front face, reciprocal to said camera 68 and said gesture sensor 70.

Referring to FIGS. 7 and 8, said releasable front cover 130 is a plastic injection moulding which comprises; an opening in the top face 131 reciprocal to said protruding bezel element 112 and an opening or indent 132 in the bottom face reciprocal to said protrusion 116. Said front cover 130 is moulded in transparent plastic and its inner surface is painted, with areas 133, 134 reciprocals to said chassis cover openings 119, 120, and area 135 reciprocal to said chassis infill panel 114 masked to remain transparent.

As best shown in FIG. 11, said front cover 130 is releasably attached to and from the said device main unit 125 by forcing the resilient top and bottom walls of the front cover 130 over said bezel element 112 and said protrusion 116.

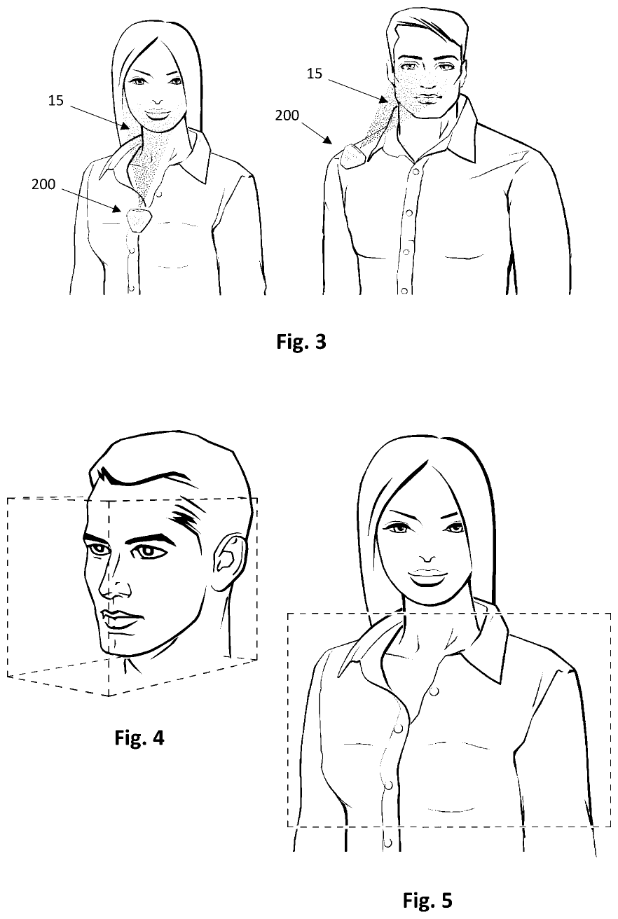

As shown in FIGS. 1, 2, and 6, said mounting element 150 has a plastic frame 151 with neodymium magnets 152 fixed thereon which co-act with said magnets 20 of said device main unit 125, coming into attractive contact to releasably sandwich a thickness of clothing for attachment of the device as shown in FIG. 2 to a wearer's upper body area shown in FIG. 5, with two preferred locations depicted in FIG. 3.

Preferably, as shown in FIG. 7, the battery 66 within the device main unit 125 is recharged by use of a magnetic contact charging cable 180 which contacts said charging port 67. During charging said LEDs 69 are activated to illuminate various colours and portions of said clear infill panel 114 to indicate device status.

In this first preferred embodiment said biocide fluid 14 is a 5% solution of Triethylene Glycol in distilled water, but any of said preferred biocides may be used.

### A Second Preferred Embodiment

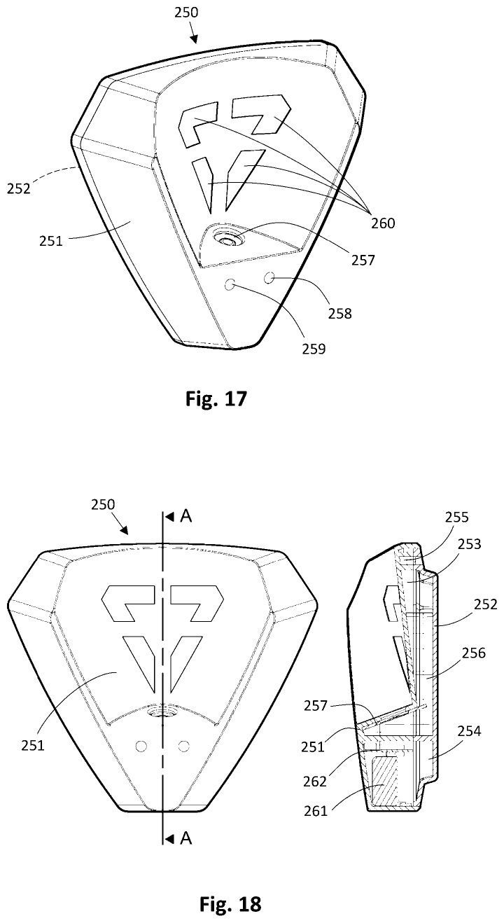

A second form of the device 250 shown in FIGS. 17, and 18, comprises; a front plastic injection moulding 251 and a rear plastic injection moulding 252 which are ultrasonically welded together to form a device body that provides a biocide fluid reservoir 253, an electronics compartment 254, and an upper rear cavity 255 for fixedly capturing various removable location means.

Said biocide fluid reservoir 253 contains a majority of a biocide fluid 256 above a piezoelectric vibrating mesh transducer 257 so that gravity may keep said biocide fluid 256 in contact with the underside of said piezoelectric vibrating mesh transducer 257.

Further, in this preferred embodiment, the front injection moulding provides status LEDs 258, on-off button 259 and clear portions 260 so that the level of the biocide fluid 256 may be visually checked by the wearer.

Said electronics compartment 254 contains a battery 261 and an electronic circuit and components 262 for the operation of the device; which components may include at least one of said components of said first embodiment 200.

In this preferred form the piezoelectric vibrating mesh transducer shoots larger ≈50 μm droplets; having a bias for inertial impact with pathogen containing respiratory droplets which may be in the wearer's airspace and which may have deposited on the wearer's face.

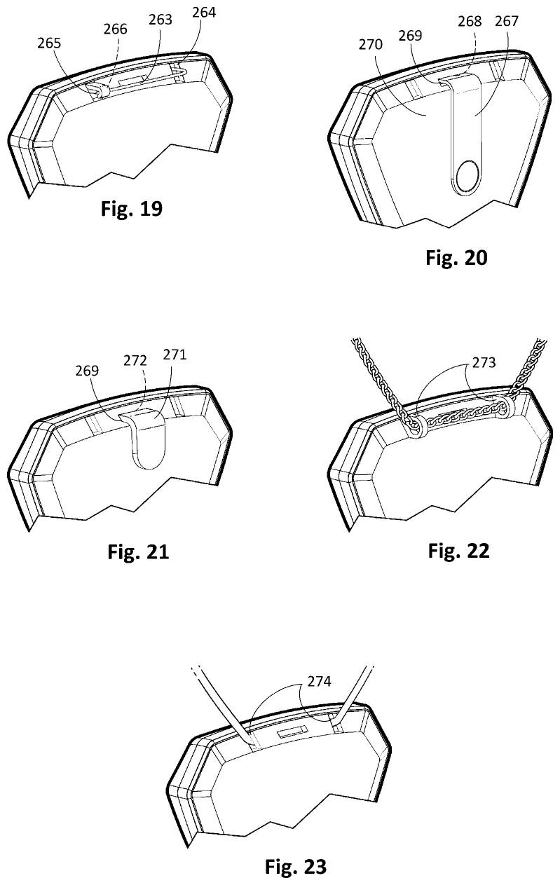

As shown in FIGS. 19, 20, 21, 22, and 23, this second form has various means for removably locating the device on the upper portion of the wearer's body shown in FIG. 5. These means include at least one of (a) a clothing pin formed of resilient metal wire having one end pivotable or flexibly captured behind an opening 264 in said upper rear cavity 255 and having a pointed end 265 which is passed through a thickness of clothing and releasably captured in an undercut area 266 shown in FIG. 18, (b) a clip formed of resilient material (metal or plastic) having an elongate body 267 and a top end 268 captured behind an opening 269 in said upper rear cavity 255, said elongate body 267 being free to flex outwardly so as to resiliently capture a thickness of clothing between said elongate body 267 and a back surface 270 of the device, (c) a downwardly oriented elongate member 271 having a top portion 272 captured behind said opening 269 in said upper rear cavity 255, said elongate member 271 being used to hook over a thickness of clothing at the neckline or pocket of a garment, (d) a jewellery chain or necklace cord passed through an eyelet 273 or a pair of openings 274 in said upper rear cavity 255 to allow hanging of the device 250 around the wearer's neck.

It is to be appreciated that any one of said removable location means may be used for any embodiment of the present invention.

In this second preferred form, a Cinnamomum zeylanicum solution is the preferred biocide, however, any of said preferred biocides may be used.

### A Third Preferred Embodiment

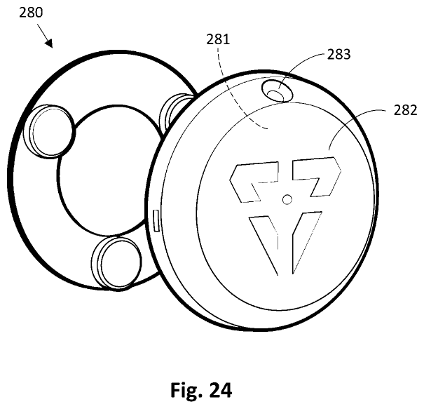

A third form of the invention 280 shown in FIG. 24, has the functionality of said first and second preferred forms, and comprises; a chassis-reservoir 281 a chassis cover (not visible), and releasable front cover 282 of a generally circular shape. This form comprises; one piezoelectric vibrating mesh transducer which generates and simultaneously shoots ≈5 μm droplets through an opening 283 in the top of the device.

Further, as shown in FIG. 13, the reservoir of any of the aforesaid embodiments, may have a biocide fluid filling port 284 with integral air venting which comprises; a hollow boss 285 integral with the rear wall of said chassis-reservoir; a valve element 290 having an opening 291 shaped to accept a Luer-slip type tapered nozzle 26 and which is slidably mounted within said hollow boss 285 and biased to a closed position by a coil spring 292 which forcibly seats an O-ring 293 in an inner annular recess 286 at the end of said hollow boss 285.

Said coil spring 292 is captured between an inward annular flange 287 of said hollow boss 285 and an outward annular flange 294 of said valve element 290. Said valve element 290 is captured within the hollow boss 285 by said O-ring 293 seated in an annular groove 295, and has holes 296 to allow biocide fluid ingress, and slotted holes 297 to allow air egress when the coil spring 292 is fully compressed by forceful insertion of a Luer-slip type nozzle 26.

As shown in FIG. 13, said filling port 284 is spring-loaded closed and sealed by forceful seating of the O-ring 293 in said inner annular recess 286 at the end of said hollow boss 285.

When filling, said nozzle 26 is snugly fitted into said opening 291 pushed inward and said valve element 290 slides inward and said coil spring 292 is fully compressed said O-ring 293 is unseated and said holes 296 are free to ingress biocide fluid and said slotted holes 297 are free to egress air.

It will be appreciated that immediately upon withdrawal of said nozzle said spring returns the valve element to the closed position as shown, and the O-ring 293 is forcibly re-seated in said inner annular recess 286 and the refill port closed.

It is to be appreciated that said biocide fluid filling port 284 with integral air venting may be used in any embodiment of the present invention. It will be appreciated that said filling nozzle may be of any practicable shape.

It will be appreciated that the device is preferably a ‘smart’ wearable personal protective device, however, various forms of the device, without a mobile device app, without AI, and having few or no sensors, although less effective, are also anticipated. For example, the wearable personal protection device may have as a simplified form of said electronics assembly, with a simple on/off button (not shown) linked to said piezoelectric pulse generator, high voltage generator, and charging circuit (not shown), enabling the timed discharge of said biocide fluid.

Furthermore, while the foregoing wearable personal protection device is primarily intended for the inactivation of airborne pathogens, it will be appreciated that said wearable personal protection device will also be used to inactivate pathogens on the wearer's face and the wearer's respiratory system surfaces; which pathogens may come to be on said surfaces by other than airborne route.

Modifications and variations such as would be apparent to the skilled addressee are considered to fall within the scope of the present invention.

1. A wearable personal protection device for protecting a wearer against airborne pathogens comprising:
a battery-powered electronic circuit and components for controllable operation of at least one piezoelectric vibrating mesh transducer which is configured to controllably intermittently simultaneously generate and shoot droplets of a biocide fluid into the wearer's proximal airspace to contact and inactivate at least some of the pathogens which may be present;
a reservoir for containing the biocide fluid therein; and
a fluid delivery apparatus for conveying said biocide fluid to an uptake location of said piezoelectric vibrating mesh transducer; and
at least one attachment element adapted for removable attachment of the device to a wearer or the wearer's garment.

2. The wearable personal protection device of claim 1 wherein said fluid delivery apparatus is chosen from a (a) wicking element, (b) pump, and (c) gravity mechanism for conveying said biocide fluid to an uptake location of said piezoelectric vibrating mesh transducer.

3. The wearable personal protection device of claim 1 wherein said at least one attachment element is chosen from:
(a) a magnet and a co-attractive element, (b) a clothing pin, (c) a resilient clip, (d) a hanging hook, (e) a necklace, and (f) a neck string.

4. The wearable personal protection device of claim 1 wherein at least a portion of said biocide fluid droplets are shot into the wearer's airspace such that said droplets enjoin the wearer's inspired airstream to travel into the wearer's respiratory system to contact with and inactivate at least a portion of pathogens which may be in the said inspired air stream.

5. The wearable personal protection device of claim 1 wherein at least a portion of said biocide fluid droplets are shot at the face and/or mucosa of the wearer's face so at least some of said droplets strike and inactivate at least a portion of said pathogens which may be thereon, and which may subsequently deposit thereon.

6. The wearable personal protection device of claim 1 wherein at least a portion of said biocide fluid droplets enter the wearer's respiratory system and deposit on surfaces therein to inactivate at least a portion of said pathogens which may have deposited thereon, and which may subsequently deposit thereon.

7. (canceled)

8. (canceled)

9. The wearable personal protection device of claim 1 wherein at least a portion of said biocide fluid droplets are given either (a) a positive electrical charge, or (b) a negative electrical charge, to increase collision-coalescence efficiency with neutral or oppositely charged droplets and particles.

10. The wearable personal protection device of claim 1 wherein said biocide fluid droplets contain (a) Triethylene glycol, (b) H2O2, (c) Propylene glycol, (d), an aromatic oil, (e) any suitable biocide, or (f) combinations thereof, diluted to a level known to be safe for use by humans while remaining efficacious for inactivation of at least some pathogens contacted.

11. The wearable personal protection device of claim 1 comprising:
a central processing unit chip (CPU);
a wireless receiver and transmitter component, which communicates with and detects the proximity of wireless devices,
wherein the electronic circuit co-acts with said CPU and said wireless receiver and transmitter component to allow (a) setting of device status, (b) control of device status, (c) viewing of device status, (d) viewing notifications and reports, or (e) combinations thereof with a software application (app) on a mobile device.

12. (canceled)

13. The wearable personal protection device of claim 11 a haptic feedback component which is used to alert the wearer, via different pulses, of (a) biocide fluid level, (b) battery charge level, (c) estimated pathogen levels, (d) various statuses of the device, (e) likely presence of specific pathogens and irritants or a class of pathogen or irritant, or (f) combinations thereof.

14. The wearable personal protection device of claim 11 comprising:
a gyroscope-accelerometer sensitive to the wearer's chest movements and adapted to enable synchronization of bursts of said biocide fluid droplets from said piezoelectric vibrating mesh transducer with (a) the wearer's exhalation, (b) wearer's inhalation, (c) the pause between the wearer's inhalation and exhalation, or (d) combinations thereof.

15. The wearable personal protection device of claim 11 comprising:
artificial intelligence software (AI) which uses data from said electronic components to autonomously operate the personal protection wearable so as to facilitate customizing protection to the physiology, breathing rate, tidal volume, and/or behaviour of the wearer and usage of the minimum quantity of biocide fluid needed to inactivate a majority of pathogens computed to be likely present in the wearer's said proximal breathing air space.

16. The wearable personal protection device of claim 15 comprising:
(a) a barometric pressure sensor, (b) a temperature and humidity sensor, or (c) combinations thereof, which supplies data to said (a) CPU, (b) app, (c) AI software, or (d) combinations thereof, which uses the data to determine the probability of pathogen presence in said proximal breathing air space, and to determine the likely viability of a specific pathogen or type, said viability being known to be dependent on barometric pressure, air temperature and humidity, said air barometric pressure, temperatures and humidity levels relative to at least some of known pathogens being made assessable by said CPU, app, AI software, or combinations thereof.

17. The wearable personal protection device of claim 15 comprising:
a camera component, to detect proximal people;
a microphone, to allow the said wearable device to respond to the sound of coughs, sneezes and speech of said proximal people, which sounds are known to coincide with emissions of respective levels of respiratory mucous droplets which are a source of pathogens;
said components, together with data from other said sensors support computation of risk level and computation of an effective immediate protective response by said wearable personal protection device, and as decided by either the CPU or AI the device takes the following action(s): (a) generates biocide fluid droplets commensurate with computed threat level, (b) generates haptic feedback warnings of various pulses signalling signaling various states, (c) issues a notification via said wirelessly linked mobile device app, or (d) combinations thereof.

18. The wearable personal protection device of claim 11 wherein said app is used on a mobile device and said app uses data from (a) a global position system component (GPS), (b) mobile device location by triangulation of cell tower locations, (c) wi-fi location positioning, or (d) combinations thereof, to compute the wearer's geographical location, and together with data from other sensors which include (a) a camera, (b) a gesture sensor (c) barometric pressure sensor, (d) humidity and temperature sensor, (e) wireless transmitter and receiver, (f) an accelerometer-gyroscope, (g) a microphone, or (h) combinations thereof, whereby the pathogen threat level may be computed and communicated to the wearer by (i) a haptic feedback signal on said personal protection wearable, (ii) an app-initiated sound emitted from said wearable, said mobile device, or both, (iii) images displayed by said app on said mobile device, (iv) text displayed by said app on said mobile device, (v) an audio speech virtual assistant on said mobile device, or (vi) combinations thereof;
and the wearable will also automatically generate biocide fluid droplets, or not generate biocide fluid droplets commensurate with said computed threat level.

19. The wearable personal protection device claim 15 wherein data recorded by the wearable is used by (a) said app, (b) said AI software, or (c) combination thereof to prepare periodic threat and threat mitigation action reports, which the wearer may access and view displayed by the said app on said mobile device;
said prepared reports enable the wearer to learn of high threat times, locations and situations to allow for proactive minimization of exposure to high pathogen loads.

20. The wearable personal protection device of claim 1, wherein said biocide fluid reservoir includes a biocide fluid filling port comprising;
a hollow boss integral with a wall of said biocide fluid reservoir;
a hollow form with an open end shaped to accept a Luer-slip type or otherwise nozzle therein and having a closed end, said hollow form being slidably mounted within said hollow boss;
further comprising;
a coil spring captured between an annular ring at said open end of said slidably mounted hollow form and an opposite annular ring near the end of said hollow boss;
said slidably mounted hollow form and said spring is captured within said hollow boss by an O-ring in an annular trench;
said slidably mounted form further having at least one inwardly directed hole in its wall to allow biocide fluid ingress to said reservoir, and at least one outwardly directed hole to allow air venting from said reservoir during biocide fluid ingress when the spring is fully compressed by forceful insertion of said filling nozzle;
closure of said ingress hole and said air venting hole is effected when a nozzle is removed and said spring resiliently returns said slidably mounted hollow boss to a rest position;
and whereby said O-ring is simultaneously forcedly re-seated by said spring in an internal annular recess at the open end of said hollow boss.

21. The wearable personal protection device of claim 1 wherein said biocide fluid reservoir includes a biocide fluid filling port comprising:
a tapered orifice having at the narrow end a fluid egress prevention valve;
said tapered orifice sized to accept a reciprocal said nozzle fitted to a separate biocide fluid refill pack; which refill pack co-acts with said orifice to effect fast, easy refill of said personal protection wearable.

22. (Canceled)

23. The wearable personal protection device of claim 1 further comprising:
at least one ring-shaped electrode positioned above said piezoelectric vibrating mesh transducer;
a ground electrode in said biocide fluid reservoir, said ring-shaped electrode and said ground electrode adapted to impart a positive or negative electrical charge to at least some of the biocide fluid droplets generated and propelled by said piezoelectric vibrating mesh transducer, which increases the collision-coalescence rate of said droplets with (a) respiratory mucus droplets containing pathogens, (b) pathogens not contained in mucus droplets, or (c) combinations thereof, wherein mucus droplets and pathogens are either negatively or positively charged.

24. (canceled)

25. The wearable personal protection device of claim 11 wherein said app uses said mobile device to wirelessly access a database that supplies information relative to the locations of known sources of pathogens and uses said information together with other data to effectively operate the said wearable personal protection device.

26. The wearable personal protection device of claim 11 further comprising at least one motion sensor component, so that on and off and various functions of the device may be controlled by hand gestures of the wearer communicating with said motion sensor.

27. The wearable personal protection device of claim 11 further comprising at least one microphone component and an audio processor for sound and/or speech recognition, which enables (a) on and off control, (b) various functions, or (c) combinations thereof, by the wearer's utterances.

28. (canceled)

29. The wearable personal protection device of claim 1 further comprising a releasable front cover, comprising:
at least one aperture or indent in a first wall and at least one aperture or indent in a second wall that is opposite the first wall, which apertures or indents are reciprocal to short protrusions in the body of said personal wearable device; said walls being formed of sufficiently resilient material to allow said walls to flex outward from said protrusions and flex back to releasably capture said protrusions in said apertures or indents when said front cover is forced against said protrusions; whereby the wearer may release the cover and replace it with another or differently designed cover.

30. (canceled)

31. (canceled)

32. (canceled)

33. (canceled)

34. (canceled)

35. A method of personal protection from airborne pathogens, comprising the steps of:
(i) providing an assembly of a wearable comprising a biocide fluid in a reservoir, a battery-powered electronic circuit, and at least one electronically powered piezoelectric vibrating mesh transducer to shoot biocide fluid droplets;
(ii) providing a fluid delivery apparatus for conveying said biocide fluid to an uptake location of said piezoelectric vibrating mesh transducer;
(iii) providing at least one attachment element adapted for removable attachment of the wearable to a user;
(iv) orientating said assembly such that said assembly when activated controllably shoots said biocide fluid droplets into the proximal breathing air space and face of the wearer;
(v) whereby said biocide fluid droplets may contact with and inactivate pathogens in the following manner(s): (a) collision within said breathing air space, (b) interception by Brownian motion and/or electrical attraction in the wearer's inspired air, (c) deposition on at least a portion of surfaces of the wearer's respiratory system, (d) deposition on at least a portion of the mucosa of the wearer's face, or (e) combinations thereof.
